(19) United States
(12) Patent Application Publication
(10) Pub. No.: US 2025/0319545 A1
(43) Pub. Date: Oct. 16, 2025

(54) RAPID AUTOMATED SWAB CUTTING LASER SYSTEMS AND METHOD

(71) Applicant: Georgia Tech Research Corporation, Atlanta, GA (US)

(72) Inventors: Tabitha Rosenbalm, Atlanta, GA (US); Roman Aranda, Atlanta, GA (US); Michael E. Cato, Atlanta, GA (US); Nicholas Colby Speller, Atlanta, GA (US)

(21) Appl. No.: 18/868,887

(22) PCT Filed: May 26, 2023
(86) PCT No.: PCT/US2023/067559

§ 371 Date: Nov. 25, 2024

Related U.S. Application Data
(60) Provisional application No. 63/365,338, filed on May 26, 2022.

(51) Int. Cl. B23K 26/08; B23K 26/03; B23K 26/16; B23K 37/04

(52) U.S. Cl. CPC B23K 26/0869; B23K 26/032; B23K 26/16; B23K 37/0426

(57) ABSTRACT

An exemplary embodiment of the present disclosure provides a system for processing swabs. The system can include a cutting laser configured to emit a light beam and a swab sleeve. Swab sleeve can include a hollow body configured to house a swab, an aperture disposed on the hollow body in a position, such that when the swab is positioned proximate the aperture, the swab is exposed to the light beam of the cutting laser when the cutter laser emits the light beam. Swab sleeve can further include a seal configured to hold the swab in the swab sleeve.

### CROSS-REFERENCE TO RELATED APPLICATIONS

This application claims the benefit of U.S. Provisional Application Ser. No. 63/365,338, filed on 26 May 2022, which is incorporated herein by reference in its entirety as if fully set forth below.

### FIELD OF THE DISCLOSURE

The various embodiments of the present disclosure relate generally to processing of chemical and biological samples, and more particularly to high-throughput, automated chemical and biological sample processing systems and methods.

### BACKGROUND

Swabs are commonly used to collect biological and chemical samples. Following collection, the swabs are then processed and analyzed in order to obtain a diagnostic result, which can require the addition of reagents to the swab or vice versa. Currently, swab tips are cut off from the end of swabs, for example with a scalpel. This is a time and labor-intensive process that introduces the potential for human error and cross contamination. Furthermore, this process introduces the risk of mixing up and misidentifying swabbed samples, for example, if an operator misplaces the cut end of a swab in a well identified as having the cut end of a different swab, then the results of the diagnostic test will be misreported. Thus, there is a need for an accurate, high-throughput system that minimizes the potential for human error and cross contamination.

### BRIEF SUMMARY

The present disclosure relates to high throughput swab processing. An exemplary embodiment of the present disclosure provides a system for processing swabs. The system can include a cutting laser configured to emit a light beam and a swab sleeve. Swab sleeve can include a hollow body configured to house a swab, an aperture disposed on the hollow body in a position, such that when the swab is positioned proximate the aperture, the swab is exposed to the light beam of the cutting laser when the cutter laser emits the light beam. Swab sleeve can further include a seal configured to hold the swab in the swab sleeve.

In any of the embodiments disclosed herein, the system can further include an exhaust configured to evacuate particulate matter from the swab sleeve.

In any of the embodiments disclosed herein, the system can further include a carrier block including a closure configured to compress the seal of the swab sleeve. The swab sleeve can be one of a plurality of swab sleeves disposed in the carrier block.

In any of the embodiments disclosed herein, the seal can include a flexible gasket and the closure can include a clamping member, the flexible gasket configured to create an airtight seal around the swab when compressed by the clamping member.

In any of the embodiments disclosed herein, the seal can include a locking comb configured to be inserted into the carrier block and to align the swab in the swab sleeve.

In any of the embodiments disclosed herein, the carrier block can further include an exhaust duct in fluid communication with the swab sleeve.

In any of the embodiments disclosed herein, the system can further include a vacuum manifold operatively couplable to the carrier block and the exhaust and configured to allow the exhaust to evacuate particulate matter from the swab sleeve.

In any of the embodiments disclosed herein, the plurality of swab sleeves can be aligned with a plurality of wells in a well plate such that a portion of the swab cut off by the light beam from the cutting laser falls into a well of the plurality of wells.

In any of the embodiments disclosed herein, the system can further include an imager configured to capture images of the well plate to verify the portion of the swab resides in the well.

In any of the embodiments disclosed herein, the carrier block can be transparent, and the imager can be further configured to verify a position of the swab within the swab sleeve.

In any of the embodiments disclosed herein, the plurality of swab sleeves can be removable from the carrier block.

In any of the embodiments disclosed herein, the system can further include a base plate configured to set a portion of the swab to be cut by the light beam of the cutting laser at a predetermined height in the aperture.

In any of the embodiments disclosed herein, the carrier block can be transparent.

In any of the embodiments disclosed herein, the system can further include a track configured to move the carrier block relative to the cutting laser.

In any of the embodiments disclosed herein, the system can further include a track configured to move the cutting laser relative to the carrier block.

In any of the embodiments disclosed herein, the system can further include a carousel configured to move the carrier block relative to the cutting laser.

In any of the embodiments disclosed herein, the system can further include a robotic arm configured to move the carrier block relative to the cutting laser.

In any of the embodiments disclosed herein, the system can further include a scanner configured to scan an identifier on the swab.

In any of the embodiments disclosed herein, the system can further include a storage container into which the cut swab is placed.

Another exemplary embodiment of the present disclosure provides a system for processing swabs. The system can include a cutting laser configured to emit a light beam, a carrier block, an exhaust, a vacuum manifold, and a scanner. Carrier block can include a plurality of swab sleeves, an aperture through which the swab can be exposed to the cutting laser, a seal configured to hold the swab in the swab sleeve, a closure configured to compress the seal such that the seal grips the swab, and an exhaust duct in fluid communication with the plurality of swab sleeves. Exhaust can be configured to evacuate particulate matter from the carrier block. Vacuum manifold can be configured to operatively couple the carrier block to the exhaust. Scanner can be configured to scan an identifier on the swab. The system can include a track, carousel, robotic arm, or similar can be configured to move the carrier block relative to the cutting laser.

In any of the embodiments disclosed herein, the system can further include a well plate including a plurality of wells. At least a portion of the plurality of wells can be aligned with the plurality of swab sleeves such that a portion of each swab falls from a swab sleeve of the plurality of swab sleeves into a respective aligned well. An imager can be positioned to capture an image of at least one well of the plurality of wells.

In any of the embodiments disclosed herein, the carrier block is transparent, and the imager can be further configured to verify a position of the swab within the swab sleeve.

In any of the embodiments disclosed herein, the seal can include a flexible gasket and the closure can include a clamping member. The flexible gasket can be configured to create an airtight seal around the swab when compressed by the clamping member.

In any of the embodiments disclosed herein, the plurality of swab sleeves is removable from the carrier block.

In any of the embodiments disclosed herein, the system can further include a base plate configured to set a portion of the swab to be cut by the light beam of the cutting laser at a predetermined height in the aperture.

Another exemplary embodiment of the present disclosure provides a method of processing swabs. The method can include placing a swab in a swab sleeve, laser-cutting a portion from the swab, and depositing the cut portion in a container.

In any of the embodiments disclosed herein, the method can further include sealing the swab in the swab sleeve. Laser-cutting the portion form the swab can include moving the swab sleeve past a cutting laser configured to emit a light beam and exposing, through an aperture disposed on the swab sleeve, the swab to the light beam.

In any of the embodiments disclosed herein, placing the swab in the swab sleeve can include inserting the swab into the swab sleeve until the swab rests against a base plate configured to set the swab at a predetermined height in the aperture corresponding to a portion of the swab to be cut by the cutting laser.

In any of the embodiments disclosed herein, method can further include evacuating particulate matter from the swab sleeve.

In any of the embodiments disclosed herein, moving the swab sleeve past the cutting laser can include translating a carrier block including the swab sleeve. Evacuating particulate matter from the swab sleeve can include placing a vacuum manifold on the carrier block, the vacuum manifold configured to establish operative communication between an exhaust and the carrier block. In any of the embodiments disclosed herein, translating the carrier block can be done on a track, on a carousel, with a robotic arm, or similar.

In any of the embodiments disclosed herein, the swab sleeve can be one of a plurality of swab sleeves disposed in the carrier block, and the container can be a well plate including a plurality of wells, at least a portion of the plurality of wells aligned with the plurality of swab sleeves.

In any of the embodiments disclosed herein, the method can further include passing the carrier block past an imager and capturing, with the imager, an image of the cut portion.

In any of the embodiments disclosed herein, the method can further include passing an imager past the carrier block and capturing, with the imager, at least a portion of at least one of the carrier block, well plate, and swab sleeve.

Another exemplary embodiment of the present disclosure provides a carrier block for use in a swab processing system. The carrier block can include a top portion configured to create a seal with a vacuum manifold, a base portion, a plurality of lumens extending from the top portion to the base portion and configured to receive a plurality of removable swab sleeves, a plurality of exhaust ducts, and a clamping member configured to hold a plurality of swabs in the plurality of removable swab sleeves and to create a seal around said swabs. The carrier block can be configured to be moved on a track. Each of the plurality of exhaust ducts can be coupled to the plurality of lumens.

In any of the embodiments disclosed herein, the carrier block can further include a lock configured to hold at least one removable swab sleeve in the carrier block.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1A provides a perspective view of a system for processing swabs, in accordance with an exemplary embodiment of the present invention.

FIG. 1B provides a top view of a system for processing swabs, in accordance with an exemplary embodiment of the present invention.

FIG. 2A provides a perspective view of a carrier block with swabs being cut by a laser and deposited in a well plate, in accordance with an exemplary embodiment of the present invention.

FIG. 2B provides another perspective view of a carrier block with swabs being cut by a laser and deposited in a well plate, in accordance with an exemplary embodiment of the present invention.

FIG. 3A provides a top view of a carrier block with swabs therein, in accordance with an exemplary embodiment of the present invention.

FIG. 3B provides top views of seals sealing a swab, in accordance with an exemplary embodiment of the present invention.

FIG. 3C provides a front view of a carrier block with swabs to be cut by a laser resting at a predetermined height on a baseplate, in accordance with an exemplary embodiment of the present invention.

FIG. 4A provides a side view of a carrier block with swabs to be cut by a laser, in accordance with an exemplary embodiment of the present invention.

FIG. 4B provides a cross section of the carrier block of FIG. 4A.

FIG. 5 a perspective view of a carrier block with swabs being cut by a laser and deposited in a well plate, in accordance with an exemplary embodiment of the present invention.

FIG. 6 provides a perspective view of a quality control imager verifying placement of a cut portion of a swab, in accordance with an exemplary embodiment of the present invention.

FIG. 7A provides a perspective view of a vacuum manifold, in accordance with an exemplary embodiment of the present invention.

FIG. 7B provides a top view of a vacuum manifold, in accordance with an exemplary embodiment of the present invention.

FIG. 8A provides a top view of a cut swab, in accordance with an exemplary embodiment of the present invention.

FIG. 8B provides a top view of cut swabs, in accordance with an exemplary embodiment of the present invention.

FIG. 8C provides a perspective view of a swab storage system, in accordance with an exemplary embodiment of the present invention.

FIG. 9 provides a flowchart of a method of processing swabs, in accordance with an exemplary embodiment of the present invention.

FIG. 10A provides a flowchart of a method of processing swabs, in accordance with an exemplary embodiment of the present invention.

FIG. 10B provides a flowchart of a method of processing swabs, in accordance with an exemplary embodiment of the present invention.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

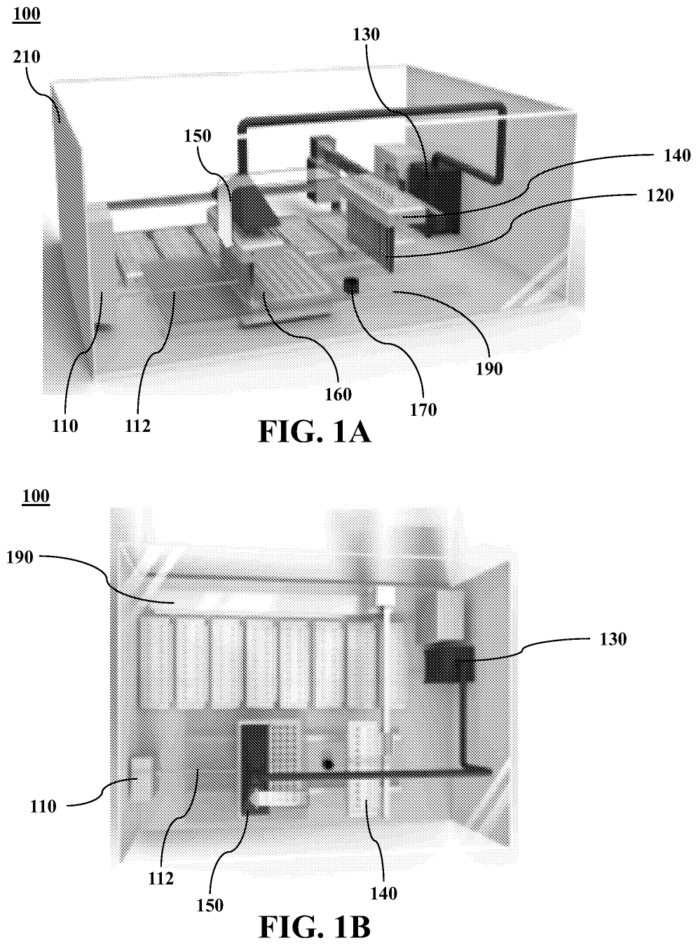

FIGS. 1A-1B show a system 100 for processing swabs 10. The system 100 can include a cutting laser 110 configured to emit a light beam 112 and a swab sleeve 120. Swab sleeve 120 can include a hollow body 122 configured to house a swab 10, an aperture 124 disposed on the hollow body 122 in a position, such that when the swab 10 is positioned proximate the aperture 124, the swab 10 is exposed to the light beam 112 of the cutting laser 110 when the cutter laser 110 emits the light beam 112. Swab sleeve 120 can further include a seal 126 configured to hold the swab 10 in the swab sleeve 120. In some embodiments, aperture 124 is not pre-formed in the swab sleeve 120. In some embodiments, aperture 124 can be formed as a result of the light beam 112 cutting the aperture 124 into the swab sleeve 120, for example if the swab sleeve 120 is made of a material that facilitates said cutting, such as paper or cardstock. Swab sleeve 120 can further include a reinforced back portion to protect the rest of the system 100 from damage by light beam 112.

In any of the embodiments disclosed herein, the system 100 can further include an exhaust 130 configured to evacuate particulate matter from the swab sleeve 120.

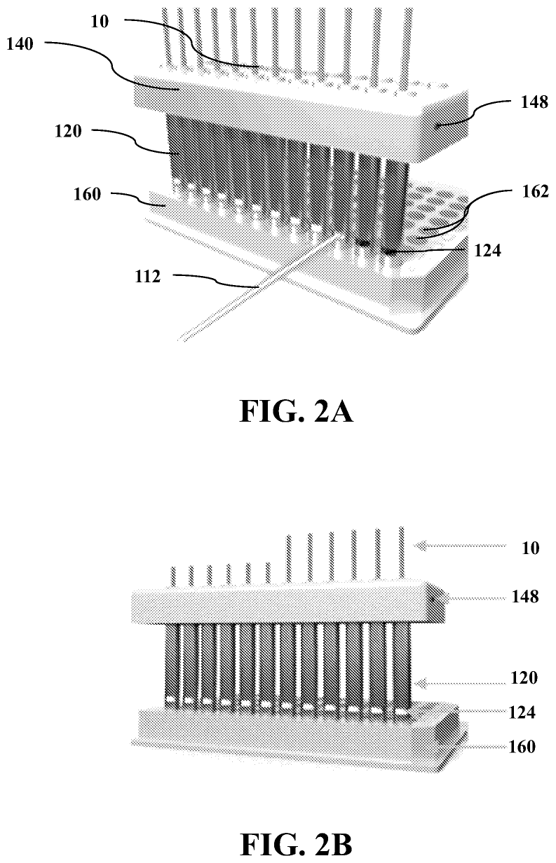

As shown in FIGS. 2A-2B, the system 100 can further include a carrier block 140 including a closure 142 configured to compress the seal 126 of the swab sleeve 120. The swab sleeve 120 can be one of a plurality of swab sleeves 120 disposed in the carrier block 140.

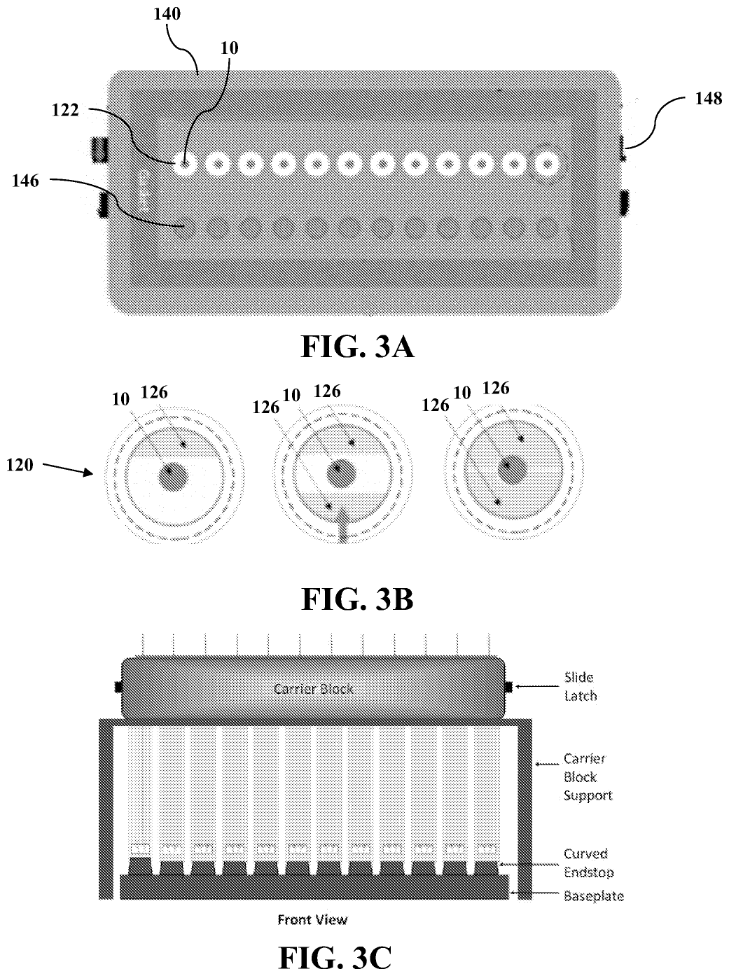

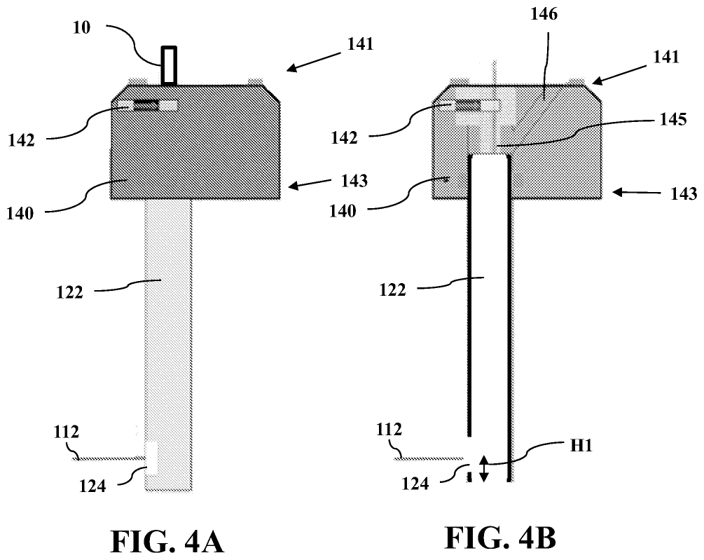

FIGS. 3A-3C show the carrier block 140 in more detail. The seal 126 can include a flexible gasket and the closure 142 can include a clamping member 144, the flexible gasket configured to create an airtight seal around the swab 10 when compressed by the clamping member 144. All components and subcomponents of system 100 can be contained in an enclosure 210. FIG. 4A shows a side view of carrier block 140. As seen in the cross section of FIG. 4A shown in FIG. 4B, the carrier block 140 can further include an exhaust duct 146 in fluid communication with the swab sleeve 120. Exhaust 103 can be configured to evacuate particulate matter from the enclosure 210.

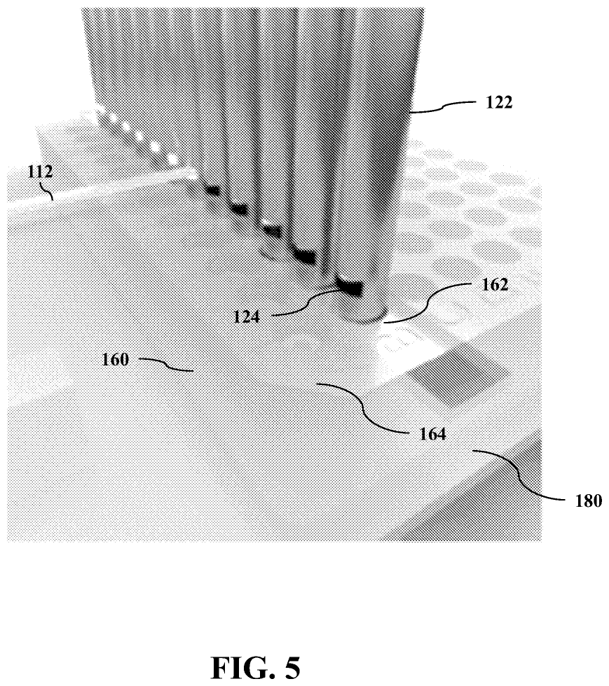

The system 100 can further include a base plate 180, shown in FIG. 5, configured to set a portion of the swab 10 to be cut by the light beam 112 of the cutting laser 110 at a predetermined height H1 in the aperture 124. System 100 can further include a plate shield 164 to further isolate each well of the plurality of wells 162 from one another.

In any of the embodiments disclosed herein, the plurality of swab sleeves 120 can be aligned with a plurality of wells 162 in a well plate 160 such that a portion of the swab 10 cut off by the light beam 112 from the cutting laser 110 falls into a well of the plurality of wells 162.

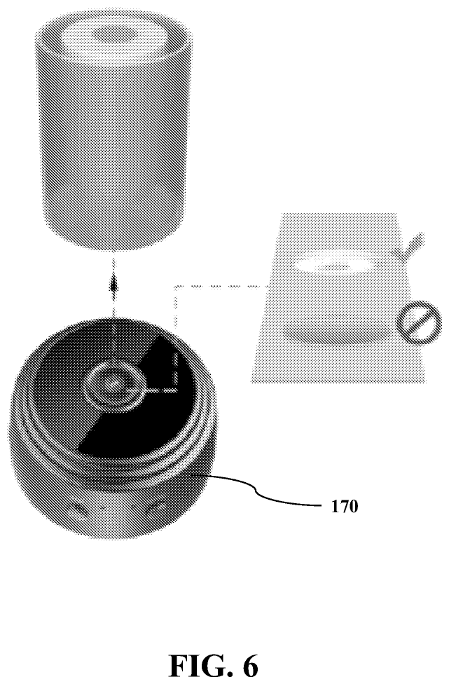

FIG. 6 shows an imager 170 configured to capture images of the well plate 160 to verify the portion of the swab 10 resides in the well.

In any of the embodiments disclosed herein, the carrier block 140 can be transparent, and the imager 170 can be further configured to verify a position of the swab 10 within the swab sleeve 120. This transparency can allow imager 170 to capture images through carrier block 140.

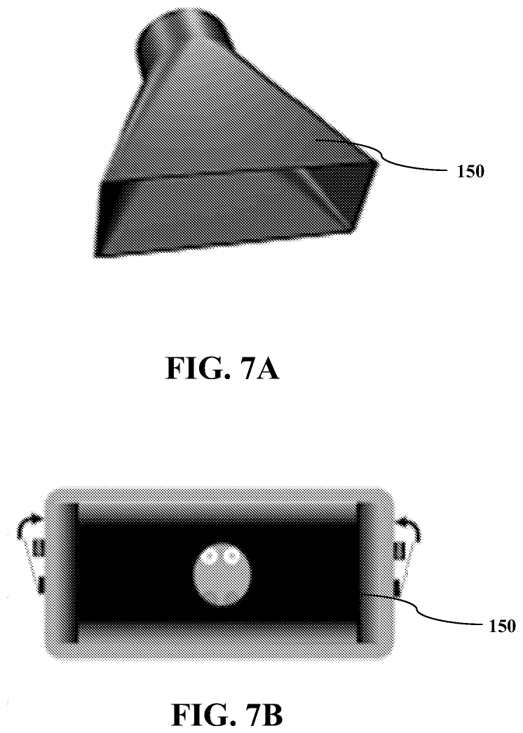

The system 100 can further include a vacuum manifold 150, shown in FIGS. 7A-7B, operatively couplable to the carrier block 140 and the exhaust 130 and configured to allow the exhaust 130 to evacuate particulate matter from the swab sleeve 120.

In any of the embodiments disclosed herein, the plurality of swab sleeves 120 can be removable from the carrier block 140. This can facilitate disposal, cleaning and/or sterilization, and placement of the swab sleeves 120 into the carrier block 140.

In any of the embodiments disclosed herein, the carrier block 140 can be transparent.

In any of the embodiments disclosed herein, the system 100 can further include a track 190 configured to move the carrier block 140 relative to the cutting laser 110.

In any of the embodiments disclosed herein, the system 100 can further include a scanner 200 configured to scan an identifier on the swab 10.

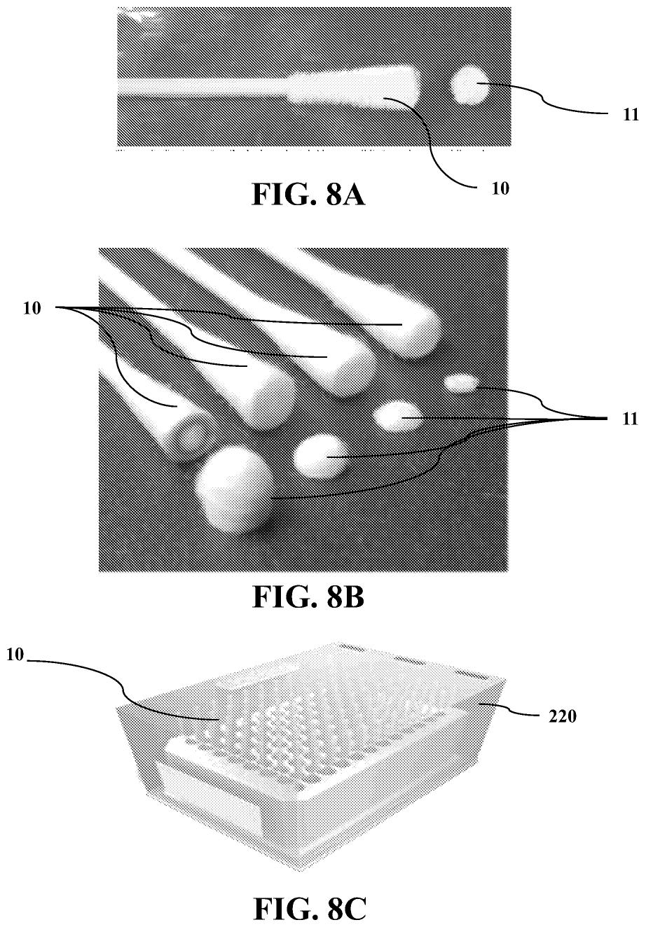

Swabs 10 cut by system 100 are are shown in FIGS. 8A-8B. Swabs 10 can be stored in the long-term storage container 220 shown in FIG. 8C.

Stated otherwise, the present disclosure provides a system 100 for processing swabs 10. The system 100 can include a cutting laser 110 configured to emit a light beam 112, a carrier block 140, an exhaust 130, a vacuum manifold 150, a scanner 200, and a track 190.

Carrier block 140 can include a plurality of swab sleeves 120, an aperture 124 through which the swab 10 can be exposed to the cutting laser 110, a seal 126 configured to hold the swab 10 in the swab sleeve 120, a closure 142 configured to compress the seal 126 such that the seal 126 grips the swab 10, and an exhaust duct 146 in fluid communication with the plurality of swab sleeves 120. Exhaust 130 can be configured to evacuate particulate matter from the carrier block 140. Vacuum manifold 150 can be configured to operatively couple the carrier block 140 to the exhaust 130. Scanner 200 can be configured to scan an identifier on the swab 10. A track 190 configured to move the carrier block 140 relative to the cutting laser 110.

In any of the embodiments disclosed herein, the system 100 can further include a well plate 160 including a plurality of wells 162. At least a portion of the plurality of wells 162 can be aligned with the plurality of swab sleeves 120 such that a portion of each swab 10 falls from a swab sleeve 120 of the plurality of swab sleeves 120 into a respective aligned well. An imager 170 can be positioned to capture an image of at least one well of the plurality of wells 162.

In any of the embodiments disclosed herein, the seal 126 can include a flexible gasket and the closure 142 can include a clamping member 144. The flexible gasket can be configured to create an airtight seal around the swab 10 when compressed by the clamping member 144.

In any of the embodiments disclosed herein, the plurality of swab sleeves 120 is removable from the carrier block 140.

In any of the embodiments disclosed herein, the system 100 can further include a base plate 180 configured to set a portion of the swab 10 to be cut by the light beam 112 of the cutting laser 110 at a predetermined height H1 in the aperture 124.

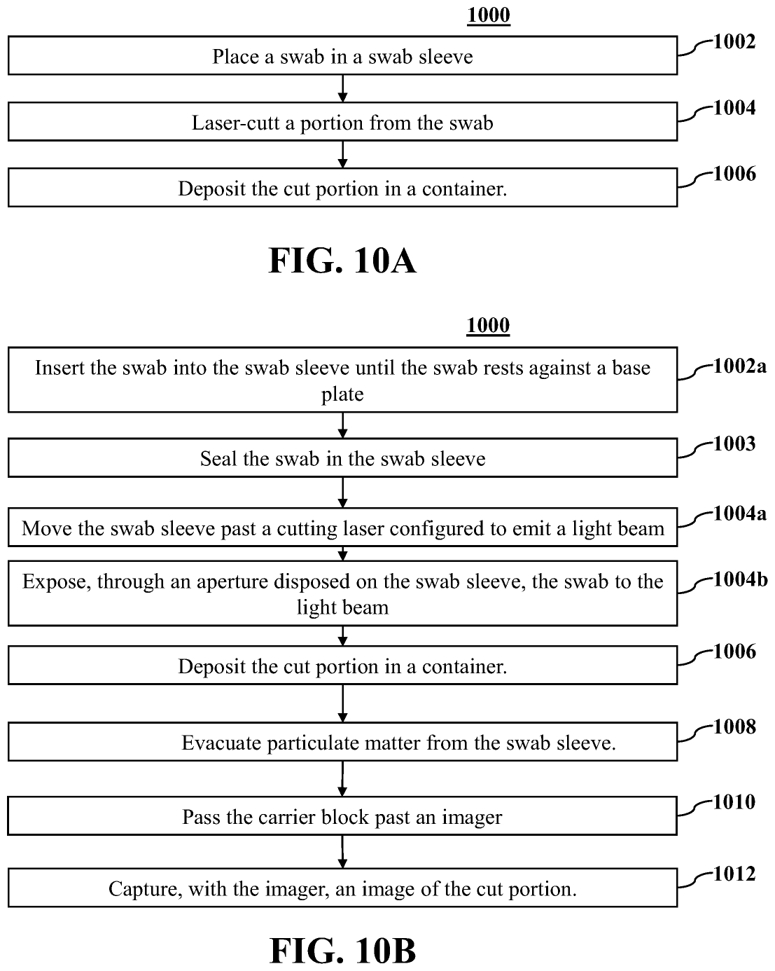

Shown in FIG. 10A, another exemplary embodiment of the present disclosure provides a method 1000 of processing swabs. The method can include placing 1002 a swab in a swab sleeve, laser-cutting 1004 a portion from the swab, and depositing 1006 the cut portion in a container.

As shown in FIG. 10B, the method 1000 can further include sealing 1003 the swab in the swab sleeve. Laser-cutting 1004 the portion form the swab can include moving 1004a the swab sleeve past a cutting laser configured to emit a light beam and exposing 1004b, through an aperture disposed on the swab sleeve, the swab to the light beam. Placing 1002 the swab in the swab sleeve can include inserting 1002a the swab into the swab sleeve until the swab rests against a base plate configured to set the swab at a predetermined height in the aperture corresponding to a portion of the swab to be cut by the cutting laser. Method 1000 can further include evacuating 1008 particulate matter from the swab sleeve. Moving 1004a the swab sleeve past the cutting laser can include translating, on a track, a carrier block including the swab sleeve. Evacuating 1008 particulate matter from the swab sleeve can include placing a vacuum manifold on the carrier block, the vacuum manifold configured to establish operative communication between an exhaust and the carrier block.

In any of the embodiments disclosed herein, the swab sleeve can be one of a plurality of swab sleeves disposed in the carrier block, and the container can be a well plate including a plurality of wells, at least a portion of the plurality of wells aligned with the plurality of swab sleeves.

In any of the embodiments disclosed herein, the method 1000 can further include passing 1010 the carrier block past an imager and capturing 1012, with the imager, an image of the cut portion.

Another exemplary embodiment of the present disclosure provides a carrier block 140 for use in a swab processing system. The carrier block 140 can include a top portion 141 configured to create a seal with a vacuum manifold 150, a base portion 143, a plurality of lumens 145 extending from the top portion to the base portion and configured to receive a plurality of removable swab sleeves 120, a plurality of exhaust ducts 146, and a clamping member 144 configured to hold a plurality of swabs 10 in the plurality of removable swab sleeves 120 and to create a seal 126 around said swabs 10. The carrier block 140 can be configured to be moved on a track 190. Each of the plurality of exhaust ducts 146 can be coupled to the plurality of lumens 145.

In any of the embodiments disclosed herein, the carrier block 140 can further include a lock 148 configured to hold at least one removable swab sleeve 120 in the carrier block 140. Lock 148 can include a latch, a sliding closure, threading, pressure fit rings, and the like.

The following examples further illustrate aspects of the present disclosure. However, they are in no way a limitation of the teachings or disclosure of the present disclosure as set forth herein.

### EXAMPLES

The example Rapid Automated Swab Cutter Laser (RASCL), an automated, contact-free DNA swab cutting system disclosed herein that uses laser-based robotics to cut swabs and deposit swab tips in standard or deep 96-well plates for subsequent DNA analysis. RASCL's automated process greatly reduces the time that qualified analysts must spend cutting swabs, increasing the throughput of reference swabs and freeing the analysts to focus on question samples. RASCL increases reproducibility, reduces operator error, and functions both in established and expeditionary laboratories. RASCL's automated processes prevents contamination and increase laboratory safety by eliminating the need for sterile scalpels.

RASCL performs contact-free cutting using a CO2 laser to cut individual swab tips at a user-selected length and minimizes debris generation. During cutting, the swabs are placed in an isolation tube with a low-flow vacuum exhaust to prevent contamination. The swab tips are then placed in the wells, and a camera and automated image analysis software confirms tip presence in each well. RASCL cuts a full plate of 96 swabs in approximately 20 minutes, reducing overall analyst labor by approximately 2 hours compared to manually slicing the swabs. RASCL employs a timed UV-light irradiation cycle and can be cleaned manually with 10% bleach or 70% isopropanol for decontamination.

RASCL provides a transformational capability at local, state, federal, and military forensic laboratories by increasing capacity to process reference samples and accelerating the generation of DNA profiles. RASCL ultimately enables a greater number of associations to unknown samples, reduce current forensics backlogs, and enhance the ability of forensic analysts to identify persons of interest.

Rapid Automated Swab Cutter Laser (RASCL) is an automated, contact-free DNA swab cutting system that uses an automated CO2 laser, such as the Keyence ML-Z9600, and robotic systems to cut and deposit swab tips into 96-well plates for downstream DNA analysis. RASCL's automated process greatly reduces the time that qualified analysts must spend cutting swabs, increasing the throughput of reference swabs and freeing the analysts to focus on question samples. RASCL's automated processes minimizes contamination and increases laboratory safety by eliminating the need for sterile scalpels.

RASCL's contact-free cutting eliminates the need for changing, decontaminating, and disposing sterile surgical blades. The 30 W ML-Z9600 laser is tunable, has a ˜150 μm beam spot size, and uses standard 120 V, 60 Hz electrical power. RASCL optimizes beam characteristics to cut each type of swab while minimizing debris generation. The laser and controller, power supply, motion control system, exhaust system, and enclosure fit into a 2′×3′×4′ footprint. RASCL reduce user involvement to: scanning swab barcodes for LIMS information, loading swabs into one of 8-12 slot marked carrier blocks with attached isolation tubes, with one swab loaded into each isolation tube, and latching the clamps to secure the swabs in position, placing the carrier blocks into their labelled positions inside RASCL, removing carrier blocks from RASCL into their marked spots on the loading dock/rack, releasing the side lock and unloading swabs from the marked carrier blocks and replacing into original packaging, and detaching the isolation tubes for cleaning. The isolation tubes can be sterile, metal-based reusable or plastic-based disposable tubes with a 5 mm cutting slit. RASCL can accommodate many swab types in the slide lock and latch system by compressing a silicone seal, holding all swabs in a carrier block in place. Swab loading and barcode scanning should take less than 30 minutes.

After the user inserts the swab loader plate into RASCL, RASCL performs the automated cutting with no additional user interaction. Slices are made through the 5 mm cutting slit in the isolation tube and swab tips fall directly into a 96-well plate. For quality control, a stationary charge-coupled device (CCD) camera, or similar camera, images the well of the swab being cut (before cutting and after) from beneath the 96-well plate, and RASCL automated image analysis software confirms swab tip presence in each well. Following the 30-minute loading process, RASCL cuts 96 swabs in approximately 20 minutes, reducing overall analyst labor by approximately 2 hours compared to manually slicing the swabs. The isolation tube reduces the risk of cross-contamination during the contact-free cutting process. RASCL's low-flow vacuum exhaust, coupled with HEPA and carbon filtration, extracts fine particulate cutting debris, further minimizing risks of cross contamination. Following cutting, RASCL returns the carrier plates to their respective starting positions. To decontaminate between runs, RASCL employs a timed UV-light irradiation cycle and can be cleaned manually with 10% bleach or 70% isopropanol.

RASCL provides a transformational capability at local, state, federal, and military forensic laboratories by increasing capacity to process reference samples and accelerating the generation of DNA profiles. RASCL ultimately enables a greater number of associations to unknown samples, reduce current forensics backlogs, and enhance the ability of forensic analysts to identify persons of interest.

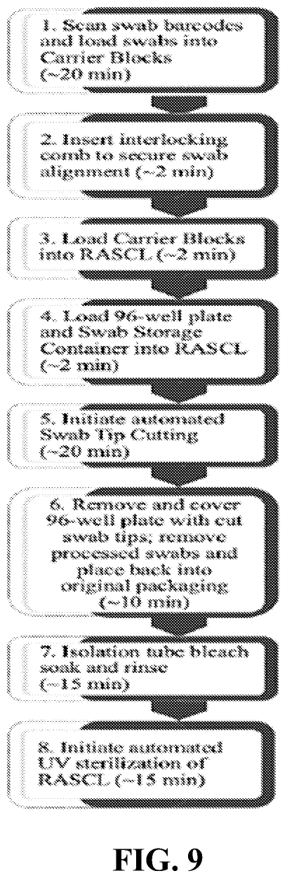

RASCL minimizes operator hands-on time for swab processing to ˜36 min (see FIG. 9). First, the operator scans each swab's barcode and load it into its swab carrier block into an isolation tube. If no barcode is present, the operator manually enters the sample's relevant laboratory information management system (LIMS) information. After loading the swab to be processed, the operator selects the swab type, swab length, and well plate type through a custom user interface (UI). This action is repeated with all swabs to be loaded with a maximum of 96 swabs. After loading swabs into a 12-slot carrier block, the block latch is closed, holding the swabs in position. Carrier blocks are loaded one at a time into their labelled positions in the RASCL instrument. The operator then ensures that the 96-well plate is loaded prior to initiating the laser cutting process. Once initiated, the cutting process completes in approximately 20 minutes. The operator removes the 96-well plate containing the processed swab tips and manually covers it for downstream processing. Processed swabs are unloaded from the carrier blocks by disengaging the latch and lock system to release the swabs and then individually placing the swabs from their marked isolation tube slot back into their original, individual sample packages. The operator can either sterilize the instrument with the automated UV-light sterilization process or manually clean the instrument with 70% isopropanol or 10% bleach, which takes a total of approximately 30 minutes to complete manual and UV sterilization, resulting in a total processing time of ˜86 minutes (see FIG. 9).

Currently, swab tips are cut by hand using sterile scalpels and forceps.

RASCL utilizes a commercial-off-the-shelf (COTS) CO2 laser, such as the 10.6 μm, 30 W, Keyence MIL-Z9600 CO2 laser, to cut individual swab tips at a user-selected length down to 1/32″. Laser-based swab processing confers several advantages compared to standard blade-based cutting, specifically: contactless cutting to minimize cross-contamination from touch point surfaces, elimination of need for disposable, interchangeable blades, operator friendly, customizable cut profiles that can be rapidly implemented, and enhanced precision and reproducibility coupled with rapid processing speeds. It has been observed that lasers can cut cotton material with more accuracy and less fibrous debris generation than a blade and are capable of micron precision cutting without cross contamination. The selected laser is tunable, has a ˜150 μm beam spot and a 300 W×300 L×42 D mm cutting field, uses standard 120 V/60 Hz AC electrical power, and has been shown to readily cut swab tips (see FIG. 8B). FIG. 8B shows ML-Z9600 laser cutting ¼″ to 1/32″ of Puritan© swab tips in under 5 milliseconds. The proposed open-source laser system is International Electrotechnical Commission IP-67-certified, which is dust-tight and waterproof. Additionally, the laser is industrial-application-ready and designed for plug-and-play implementation in custom-made architectures. The laser and its controller, power supply, motion control system, exhaust system, and enclosure will fit into the device's 2′×3′×4′ footprint. The laser system includes proprietary and open-source software (in C# and Visual Basic). Proprietary software is used for determining the proper settings and incorporates controls for the laser within the system using the open-source control software. The laser was tested at set distances for cutting for both nylon flocked and cotton swabs. The intensity, power, and cut time are optimized and set as defaults in the software. The operator only needs to select the swab type during the loading process to get precise, consistent, and accurate cuts at user-specified lengths. The enclosure adheres to FDA Class 1 laser safety requirements to meet operational safety standards.

Batches of up to 96 swabs are placed into eight individual stainless steel carrier blocks (12 swabs per carrier) for cutting and processing (see FIG. 2B). Each carrier block has 12 isolation tubes housing one swab per tube and will be numbered to match the eight numbered carrier slots in the RASCL system's loading platform. Prior to insertion of swabs, one or more racks/loading docks are placed on the counter. The carrier blocks are then placed onto the rack(s)/loading dock(s), and individual swabs are inserted from the top side of the carrier block into isolation tubes (i.e. one swab per isolation tube, see FIG. 3C).

FIG. 2B shows RASCL's Pick-and-Place System moving one carrier block (with a single row of swabs) into place for cutting. Swabs of different sizes are aligned with the interlocking comb attachment.

The loading dock(s)/rack(s) can include either one large rack or two racks that support four carrier blocks or multiple smaller racks. The proposed rack(s)/loading dock(s) can include a base plate with at least one row of curved endstops, enabling swab tip alignment with the window slot for consistent laser cutting and a rack/loading dock support which functions as a shelf to support the carrier block with attached isolation tubes (see FIG. 3C).

FIG. 3C shows a front view of rack/loading dock which illustrates the curved endstop baseplate, which supports the isolation tubes and provides an offset level for consistent height loading of swabs to position them for the window slot. The inverted egg-carton-like curves of the endstop allow the isolation tubes to rest/be supported at a lower height on the curved endstop while the peak of the curve defines the position of the swab tips relative to the bottom of the carrier block. This enables each tip in the block to have an identical and reproducible loading depth when processing various swab sizes and material types. Sufficient rack(s) are supplied to support loading 96 swabs for one instrument run.

Following placement of a rack(s)/loading dock(s) on the benchtop area for loading blocks, sterilized isolation tubes are inserted into the base of an empty carrier block (e.g., using a twist lock/luer lok mechanism). Then the carrier block with attached isolation tubes is set on the rack/loading dock and held in position by the carrier block support. The operator selects a swab to load, then scans the swab's accompanying barcode or enters the applicable information manually into the UI for the appropriate carrier block insertion position (e.g., Block A, Column/Position 1). Scanned swabs are lowered into the isolation tubes from the top of the carrier block and rest on the curved features of the base plate. The carrier plate is equipped with a bar clamp that may be locked into position from the side of the carrier plate. Swabs are loaded with the bar clamp fully open.

Following loading, a latching mechanism on the side of the carrier plate can be manually pushed closed, locking the swab handles into position by moving one arm of the bar clamp. The latching mechanism maintains the swabs' position under tension (see FIG. 3A).

FIG. 3A shows a top view of carrier block illustrating swabs inside the isolation tubes, sliding lock and latch devices for locking the swabs in place, a gasket for attachment and sealing of the vacuum manifold, vacuum suction holes/ducts directing the suctioned air away from the swabs. Closure of the swab in position results in primary exhaust airflow through the vacuum ducts. FIG. 3B shows a top view of a single isolation tube with a swab loaded, from left to right: not sealed: The bar lock is open, sealing: midpoint motion of the moving rubber seal sliding into position, and sealed: bars are locked/latched into position holding the swab tightly until the latches are released for removal.

The enclosure is outfitted with a single door to provide access to the main staging area. Once opened, loaded carrier blocks are individually lifted out of their rack/loading dock and inserted one at a time into the RASCL instrument on their corresponding labelled slots inside the system. The carrier blocks slides onto rails and is positioned by an automated system (e.g., a track or a 3-axis robotic system). A mechanical alignment pin ensures the proper orientation of the swabs in each slot.

The operator places a sterile 96-well plate per batch of swabs in the holder housed on the laser processing stage. The holder contains markings that match the row/column designation of 96-well plates. The operator visually inspects that the row/column designation of the stage and the 96-well plate match to ensure accuracy and proper chain-of-custody during laser processing. To accommodate either standard or deep 96-well sample plates, an adapter raises the regular plates to maintain a consistent laser height for all cut swabs. Automatic Pick-and-Place System.

After the swabs and carrier blocks have been installed in their designated carrier slots, the sliding access door has been closed, and the system has been configured, the carrier blocks are transferred one at a time by an automated system (e.g., a three-axis servo-driven ball lead-screw) to the laser processing stage (see FIGS. 1A and 1). The automated system aligns the position of the swab collection well plate. Once aligned, the laser activates, and the carrier plate and swab collection plate move horizontally to pass all swabs across the path of the laser beam. The laser is operated according to the user-specified inputs. Following cutting of the row, the Pick-and-Place System transports and release the processed swabs from the current carrier into the storage box.

FIG. 2A shows swab tip cutting through the individual window slot in the isolation tube. The cut swab tip then falls into the well. During cutting, the vacuum exhaustion system operates to prevent contamination. Note: The plate shield (shown in FIG. 5) is not depicted in the figure for ease of view.

A stationary camera can be positioned under the well of the swab being cut. The camera images the well prior to the cut and immediately following the cut. Following the cut, light is largely blocked by the relatively large swab cutting in the well. Each well is compared to itself before and after swab cutting. After the camera images the well, the software compares the images to confirm the presence or absence of a swab cut and update the system's software and database. The UI displays a real-time depiction of the 96-well plate status.

When a carrier block is moved to the laser processing stage, the vacuum extraction manifold is lowered by a linear actuator and coupled to the top of the carrier block. The carrier block has a row of sealed swabs held in position by a silicone bar clamp/latching system and a row of vacuum suction holes/ducts that direct flow from the isolation tubes to the exhaust system. The individual connections connect to a vacuum source (e.g., Fumex exhaust system) through the vacuum manifold (see FIGS. 7A-7B) with a vacuum hose attached to the top outlet of the vacuum manifold. The lower rectangular shape of the manifold is attached to the gasket of the carrier block (see FIGS. 3A-3B). When the carrier block reaches the cutting area, the software activates the negative pressure of the exhaust system and the vacuum manifold is lowered via a motor-driven z-axis onto the carrier block (see FIGS. 7A-7B). Suction and slight pressure allows the fumes and particulates to be drawn and trapped into the exhaust system (see FIGS. 4A-4B).

FIG. 7A shows a vacuum manifold that supplies a 2″ pipe/hose connector and a rectangular opening suitable for attachment to the gasket on top of the carrier block. FIG. 7B of carrier block with rectangular vacuum manifold affixed.

The manifold travels horizontally across the field of the laser cutting in concert with the carrier block. When laser cutting completes, first the carrier block and exhaust manifold move away from the collection plate while suction is still active. Next the suction is removed and the z-axis motor withdraws the manifold. The manifold returns to its initial position to await the next carrier block via an x-axis motor.

FIGS. 4A-4B show a side view (FIG. 4A) and a cross section of FIG. 4A (FIG. 4B). Side views of air flow for an isolation tube loaded in the carrier block and connected to the exhaust system during laser cutting. At the top, note that the slide lock pushes a rubber/silicone bar clamp into position to clamp the swab in place. Vacuum/exhaust draws air away from the swab and up to the exhaust system with directed flow to vacuum vents, which direct airflow away from the swabs. See FIG. 5 for more details about loading and securing swabs.

Laser cutting of the swabs begins after the 96-well plate has been moved into position and the vacuum extraction manifold has been coupled to the carrier block (see FIGS. 3A-3B, FIGS. 7A-7B). The carrier block and 96-well plate are then moved into the laser's field of view (FOV) so that the desired tube and swab is centered on the laser's objective. Negative pressure from the exhaust system via the vacuum ducts applies vacuum extraction to the full row of tubes on the carrier block, and the stationary laser begins cutting the swab tip. The cutting process continues for each additional swab in the carrier block and is repeated until all carrier blocks and swabs have been processed. A calibration procedure for users is carried out to ensure the robotic movement setpoints are repeatable with a high degree of precision.

The laser cuts swab tips through the isolation tube window (see FIGS. 2A-2B). Run-specific cutting parameters, based on swab material and cut height, can be applied to individual swabs or groups of swabs during the loading phase within the UI (see FIGS. 3C, 8B). To reduce the risks of carbonization of the swab cut line and sample combustion (inherent when cutting materials with CO2 lasers), the air flow, gas composition, and laser energy are pre-configured for each specific run condition and the user may not be able to change these parameters. Additionally, generated debris can be removed via the vacuum extraction manifold system. Initial testing indicates that laser parameters ranging from 59-138 inches/s and 0.8-1.6 kW/mm2 coupled with a 140 μm beam spot and multi-pass segmented cut profile safely cut the required swab tip materials. The Keyence CO2 laser can readily cut cotton (FIG. 8B) and flocked nylon (FIG. 8A) swab tips.

FIG. 8A shows cut nylon flocked swab with Keyence CO2 laser between ⅛ and 1/16″.

A camera located below the 96-well plate track checks for the presence or absence of swab tips (see FIG. 6). Images of the 96-well plate are collected and processed for quality control checks via custom imaging software. FIG. 6 shows quality control check of tip cutting via camera or optical detector. An image of the 96-well plate can be saved for chain-of-custody purposes.

The stationary camera images the well prior to the cut and after the cut and compare for contrast using open-source code that will be integrated into RASCL's software (e.g., a macro recorder with ImageJ to batch process the files). Using a light source above the well plate, any well with a cut swab is relatively dark, and empty wells are very bright. Using pre-imaged comparison swab samples of different length, the system confirms whether a cut swab tip is present. The status for all 96 wells are depicted in the UI, and detailed information is archived in the system spreadsheet and database to confirm that each swab tip has been cut and placed in the well.

After RASCL completes cutting all of the swab tips, the last carrier block is returned to its initial position. The removal process is the reverse of loading. The operator removes each carrier block individually and place it on its rack/loading dock (see FIGS. 3A-3C). Then, individual carrier blocks can be unloaded by unlocking the latch lock to release the swabs from being held. Swabs can be removed using the handle to lift each out of its respective isolation tube and to transfer it back to its original packaging. After removing all swabs from a carrier block, the isolation tubes can be twisted to release them from the carrier block for cleaning and sterilization prior to reuse. Carrier blocks and rack/loading dock baseplates should be cleaned and sterilized prior to reuse.

The isolation tubes connect to the carrier block (e.g., via twist screw top/latching system), and the carrier block's vacuum ducts withdraw air away from the swab and up to the exhaust system with directed flow to vacuum vents, which direct airflow away from the swabs to prevent cross-contamination and capture aerosolized debris (see FIGS. 4A-5). Each tube contains a slot on the laser-facing surface that enables the laser to cut the swab, while the remainder of the tube acts as a barrier to air flow, debris, and residual laser energy and scatter. This slot also acts as the inlet for one-directional air flow that routes from the cutting region to the exhaust, minimizing the potential for cross contamination. The tubes align with the wells on the 96-well plate and funnel/direct cut swabs into the appropriate wells. The proximity of the tip to the 96-well plate minimizes air flow in the adjacent wells during processing steps. As a result, the isolation tube isolates samples throughout the entire process and into final storage. After each run, isolation tubes can be decontaminated manually in a 10% bleach solution followed by DI water rinse for reuse (˜15 min), while an extra set of tubes is loaded for the next run. The isolation tubes may also be decontaminated with the automated UV sterilization function.

RASCL's low-flow vacuum extraction, coupled with HEPA and carbon filtration, extracts fine particulate cutting debris, further minimizing risks of cross contamination. The exhaust system is designed to vertically withdraw air through the carrier block tubes and away from the cut samples. Vacuum is connected for each row of samples being cut by lowering a vacuum manifold onto the gasket of the carrier block, and pressed seal comprised of either metal (e.g., surgical stainless steel or aluminum) or chemically-resistant plastic (e.g., silicone or PTFE). The silicone seal around the swab handles prevents suction of material back through the samples. The vacuum ducts withdraw air from around the swab and up to the exhaust system, which directs airflow away from the swabs (FIGS. 4A-4B). The carrier block links the rows to a primary exhaust outlet. The primary exhaust passes through a HEPA filter to capture airborne particles followed by an activated charcoal filter canister, which are part of an off-the-shelf Fumex exhaust system.

A timed UV-C light sterilization cycle decontaminates RASCL between runs. Following sample removal, the UI provides an option to activate the UV-C cycle. The UV light is located inside the top of the device enclosure and will irradiate between ˜10-20 min depending on the final instrument layout. A COTS component can be used if available. The RASCL layout also ensures that the UV light is easily accessible for replacement by a non-expert user. The isolation tubes can also be added to the instrument for sterilization during the UV-C cycle. Finally, RASCL allows for manual cleaning with 10% bleach or 70% isopropanol. The UV-C is deactivated when samples are in the system or when the access door is open, for safety.

A plate shield can be positioned between the laser's marking unit and the starting location of the empty sample well above the 96-well plate. The shield can be metal or plastic and have a horizontal slot to cut one row of swabs, while preventing cross contamination in other wells (see FIG. 5). During laser cutting, the well plate slides beneath the plate shield to protect the non-cutting wells from cross-contamination.

FIG. 5 shows a plate shield. During cutting in row B, all other rows are covered to prevent contamination. After swabs in row B are cut the isolation tubes uncouple, the well plate moves to expose row C for cutting while blocking all other rows to enable the next cutting step. The process is repeated until all swab rows are cut.

RASCL allows the operator to control the laser through a custom, easy-to-use UI that enables the operator to execute application-specific operations. The UI includes numerous real-time readback fields displaying instrument parameters and status updates. The UI enables drop-down selection of process parameters, such as swab cut size, swab material, and data storage location. TCPIP/RS232 connectivity is utilized to transmit and receive data from the laser. The data associated with commands and component readback values can be archived in a SQL database for monitoring and archiving purposes. The UI provides the operator with the ability to monitor device health and troubleshoot RASCL.

For accurate reporting and chain-of-custody, the software can record individual swab barcodes and LIMS data identifiers, such as those included in the Sample Tracking and Control Software (STACS) application. These identifiers may include the date, location, and GPS coordinates of swab collection, name of the individual associated with the swab sample, and name of the operator processing the sample batch. After the swab data are archived into the system and the cutting run has been initiated, RASCL can record the swab's location in the carrier block, presence or absence of the cut swab tip in the well, the cut height, the processed swab's location in the storage container, and other run-specific parameters. For quality control, an image of each swab tip in the well is taken and displayed in the UI. Swab information, including storage location, can be exported as an Excel-compatible spreadsheet or PDF, along with a graphical representation of the well plate and image of the wells.

The RASCL enclosure secures swab processing components, maintains mechanical alignment of the laser, and guard against external contaminants (see FIGS. 1A-1B). The enclosure complies with all safety requirements for laser and UV systems. The enclosure design meets operational constraints such as space above the instrument or aisle space. The enclosure can be UV-resistant and eliminate laser micro-cuts to maintain enclosure integrity, durability and safety. This can be accomplished by incorporating a ½″ thick, 12″ tall, bar of anodized aluminum along the wall that the laser beam faces.

FIG. 1A shows a side view of the current RASCL layout. The laser is cutting a row of swabs to the right while the other rows of the 96-well plate are protected by the plate shield. The arrow indicates carrier block and robotic arm moving into place for swab cutting.

FIG. 1A shows a top view of the current RASCL layout. The arrow indicates carrier block and robotic arm moving into place for swab cutting. View is for layout purposes.

Sample processing consumables can include the 96-well plate and aluminum foil sealing film, which are single use items. Standard 96-well plates cost between $2-4 each, deep 96-well plates cost between $9-18 each, and aluminum sealing film (such as AlumaSeal® 96 film) costs $0.72 each. Contamination control COTS consumables include the UV light, HEPA filter, and carbon pre-filter. The UV-C light should be replaced annually at an estimated price $68.50 for 365 nm, 25 W bulb and $86.50 for 254 nm, 25 W bulb depending on the final design length. The UV-C is available from government-approved vendors. Replaceable filters have been quoted at $115 per set which includes prefilter, HEPA filter, and activated carbon filter. Both items likely need to be replaced on a bi-yearly basis (dependent on usage) and can be purchased through government-approved vendors. Depending on final design of the vacuum system, the instrument may use a stack of pre-filter and HEPA filters for optimal filtration. The listed vendor costs are from online catalog pricing and do not reflect government rates.

Expected custom consumables include the isolation tubes. With the current proposed design, the reusable tubes are estimated to cost $4.95 per tube, and 96 tubes are needed for a full 96-well plate run. The isolation tubes can be used repeatedly but must be sterilized between runs. It is expected that the operator can utilize multiple reusable isolation tube sets to minimize delays between runs.

In summary, this example instrument has features and capabilities that enhance utility and usability. RASCL functions with a standard 96-well plate and a deep 96-well plate, ensuring compatibility with current downstream extraction instruments. RASCL has the ability to cut the following swab head types within the same run: cotton tip on a 6-inch wooden applicator (Puritan swabs), cotton tip on a 6-inch plastic applicator, foam tip on a 6-inch plastic applicator, cotton tip on an 8-inch wooden applicator (Puritan Jumbo Swabs), foam/wire bristle tip on a 6-inch plastic applicator, and Puritan™ PurFlock™ Ultra Flocked Swabs on a 6-inch applicator. RASCL has the capability to cut between ⅛ to ¼ of the swab tip ( 1/16″ to ⅛″) and retain the rest of the swab for re-testing purposes. The cutting sizes are adjustable so that variable cut lengths can be acquired. RASCL places the cut swab tips into a 96-well plate without any assistance or oversight from an operator. The operator is able to load RASCL, select the run, walk away, and return to a fully cut plate. The loading, processing and cutting the swabs, and unloading the swabs from RASCL requires no more than three hours. The hands-on time loading and removing swabs from RASCL will be less than 60 minutes. RASCL does not introduce cross-contamination between samples and includes a mechanism to automatically sterilize the cutting apparatus between sample runs. RASCL's sterilization process is a scientifically proven method for sterilizing items for use in short tandem repeat (STR) DNA analysis. RASCL can track all swabs through the loading, cutting, and placement in the 96-well plate. RASCL can also track the processed swabs in the storage plate, so that each swab's location is known at all times during loading, processing, and post-processing phases. RASCL possesses tracking software compatible with Microsoft Windows 10, or the current U.S. Army required operating system, McAfee™ Antivirus software, and operate on a laptop or desktop computer system. RASCL can possess software that follows Security Technical Implementation Guides (STIG), complies with the Federal Information Processing Standards (FIPS), is able to accept Microsoft patches, and is approved by the US Government. RASCL can output the post-processing file in a delimited file format, such as Microsoft Excel, a Comma Separated Values file, or Portable Document Format (i.e., .xlsx, .csv, .pdf). RASCL can include an operation and maintenance manual and the ability to facilitate I/O to an external computer. RASCL is able to withstand sterilization of the instrument's hardware and deck with common laboratory reagents such as 70% isopropanol, 10% bleach, and ultraviolet (UV) light exposure. RASCL can have dimensions that do not exceed 3′ H×2′ D×4′ L. RASCL can function on a lab benchtop (both traditional and expeditionary) that is able to contain the 3′ H×2′ D×4′ L dimensions of the instrument and be enclosed for sterility during the processing runs. RASCL is designed to incorporate COTS consumables allowing for replacement of items, replacement of consumables during the preventative maintenance, and assisting during validation efforts. RASCL can utilize electrical power that does not exceed 120 volts/10 amperes and have a power cord that protects against electrical surges to support expeditionary sites.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

1. A system for processing swabs comprising:
a cutting laser; and
a swab sleeve comprising:
a hollow body configured to house a swab; and
a seal configured to hold the swab in the swab sleeve.

2. The system of claim 1, wherein the swab sleeve further comprises:
an aperture disposed on the hollow body such that when the swab is positioned proximate the aperture, the swab is exposed to a laser of the cutting laser.

3. The system of claim 2 further comprising at least one of:
an exhaust configured to evacuate particulate matter from the swab sleeve;
a carrier block comprising a closure configured to compress the seal of the swab sleeve, wherein the swab sleeve is one of a plurality of swab sleeves disposed in the carrier block;
an imager configured to capture images; or
a base plate configured to set a portion of the swab to be laser cut at a predetermined height in the aperture.

4. The system of claim 2 further comprising:
a carrier block comprising:
a closure configured to compress the seal of the swab sleeve;
wherein:
the swab sleeve is one of a plurality of swab sleeves disposed in the carrier block;
the seal comprises a flexible gasket;
the closure comprises a clamping member; and
the flexible gasket is configured to create an airtight seal around the swab when compressed by the clamping member.

5. The system of claim 2 further comprising:
a carrier block comprising:
a closure configured to compress the seal of the swab sleeve;
an exhaust duct in fluid communication with the swab sleeve.

6. The system of claim 5 further comprising:
a vacuum manifold operatively couplable to the carrier block and the exhaust duct and configured to evacuate particulate matter from the swab sleeve.

7. The system of claim 2 further comprising:
a carrier block comprising:
a closure configured to compress the seal of the swab sleeve;
wherein:
the swab sleeve is one of a plurality of swab sleeves disposed in the carrier block; and
the plurality of swab sleeves is aligned with a plurality of wells in a well plate such that a portion of the swab cut off by a laser from the cutting laser falls into a well of the plurality of wells.

8. The system of claim 7 further comprising:
an imager configured to capture images of the well plate into which the cut off portion of the swab fell to verify the portion of the swab resides in the well.

9. The system of claim 8, wherein the imager is further configured to verify an orientation of the swab within the swab sleeve.

10. The system of claim 2 further comprising:
a carrier block comprising:
a closure configured to compress the seal of the swab sleeve;
wherein:
the swab sleeve is one of a plurality of swab sleeves disposed in the carrier block; and
the plurality of swab sleeves is removable from the carrier block.

11. A system for processing swabs comprising:
a cutting laser;
a swab sleeve comprising:
a hollow body configured to house a swab;
an aperture disposed on the hollow body such that when the swab is positioned proximate the aperture, the swab is exposed to a light beam of the cutting laser when emitted from the cutting laser; and
a seal configured to hold the swab in the swab sleeve; and
a carrier block comprising:
a closure configured to compress the seal of the swab sleeve;
wherein the system further comprises at least one of:
a base plate configured to set a portion of the swab to be cut by the light beam at a predetermined height in the aperture;
a positioner configured to change a relative position of the carrier block to the cutting laser; or
a scanner configured to scan an identifier on the swab.

12. (canceled)

13. The system of claim 11, wherein the carrier block is transparent.

14. The system of claim 11, wherein the positioner is selected from a group consisting of:
a track configured to enable the carrier block to move relative to the cutting laser;
a track configured to enable the cutting laser to move relative the carrier block;
a carousel configured to move the carrier block relative to the cutting laser;
a carousel configured to move the cutting laser relative to the carrier block;
a robotic arm configured to move the carrier block relative to the cutting laser; and
a robotic arm configured to move the cutting laser relative to the carrier block.

15.-18. (canceled)

19. A system for processing swabs comprising:
a carrier block comprising:
swab sleeves, each swab sleeve comprising:
a hollow body configured to house a swab; and
an aperture through which the swab can be exposed to a cutting laser;
a sealing system configured to hold the swabs in the swab sleeves;
a closure configured to compress the sealing system such that the sealing system grips the swabs; and
an exhaust duct in fluid communication with the swab sleeves;
an exhaust configured to evacuate particulate matter from the carrier block;
a vacuum manifold configured to operatively couple the carrier block to the exhaust; and
a scanner configured to scan an identifier on the swabs.

20.-22. (canceled)

23. The system of claim 19 further comprising:
a cutting laser;
a well plate comprising wells; and
an imager positioned to capture one or more images of at least one of the wells;
wherein at least a portion of the wells are aligned with the swab sleeves such that a portion of each respective swab falls from a respective swab sleeve of the swab sleeves into a respective aligned well of the aligned wells.

24. The system of claim 23, wherein the imager is further configured to verify a position of one or more of the swabs within respective swab sleeves.

25. The system of claim 19, wherein;
the sealing system comprises a flexible gasket;
the closure comprises a clamping member; and
the flexible gasket is configured to create an airtight seal around the swabs when compressed by the clamping member.

26.-28. (canceled)

29. A method of processing swabs comprising:
sealing a swab in a swab sleeve; and
laser-cutting a portion from the swab.

30. The method of claim 29 further comprising:
depositing the cut portion of the swab in a container;
wherein laser-cutting the portion from the swab comprises:
moving the swab sleeve past a cutting laser; and
exposing, through an aperture in the swab sleeve, the swab to a light beam from the cutting laser.

31.-36. (canceled)

37. A carrier block for use in a swab processing system comprising:
a top portion configured to create a seal with a vacuum manifold;
a base portion;
lumens extending from the top portion to the base portion and configured to receive removable swab sleeves;
exhaust ducts, a respective exhaust duct coupled to a respective lumen of the lumens; and
a clamping member configured:
to hold swabs in the removable swab sleeves; and
to create a seal around the swabs.

38. (canceled)
